(19) United States
(12) Patent Application Publication
(10) Pub. No.: US 2004/0068203 A1
(43) Pub. Date: Apr. 8, 2004

(54) Sensing pressure

(75) Inventors: Barry N. Gellman, N. Easton, MA (US); Michael Madden, Princeton, MA (US)

Correspondence Address: TESTA, HURWITZ & THIBEAULT, LLP, HIGH STREET TOWER, 125 HIGH STREET, BOSTON, MA, 02110, US

(73) Assignee: SCIMED Life Systems, Inc., Maple Grove, MN (US)

(21) Appl. No.: 10/263,395

(22) Filed: Oct. 3, 2002

(51) Int. Cl. A61B 5/103

(52) U.S. Cl. 600/587

(57) ABSTRACT

A device for measuring pressure applied to a urethra of a patient includes an elongate member with a plurality of pressure sensors disposed thereon. The device can be inserted into the urethra. Each sensor is capable of outputting a signal representative of an amount of pressure applied to the urethra at the location of that sensor when the elongate member is positioned within the urethra and pressure is applied to the urethra. The pressure can be applied to the urethra by a urethral sling. A physician can manipulate and adjust the sling based on the pressure information provided by the device.

### TECHNICAL FIELD

The present invention generally relates to sensing pressure within a tract of the body, and more particularly to sensing, measuring, and monitoring intraurethral wall pressure applied to contiguous or arrayed sensors in connection with a medical procedure for treating urinary incontinence in female patients.

### BACKGROUND INFORMATION

The surgical treatment of stress urinary incontinence generally includes locating and securing a sling or similar support structure within the body such that a region of the urethra (e.g., the mid-urethra area or the neck of the bladder) is repositioned to minimize the undesirable passage of urine resulting from conditions of stress urinary incontinence.

Current placement of urethral slings for the treatment of urinary incontinence typically requires the physician to locate the sling in a particular location beneath the urethra and to adjust the sling to an appropriate tension. Such tension adjustment is usually dependent upon the location of the sling and upon the experience of the physician. Various techniques exist for implanting a sling. Current sling tensioning methods do not involve placing sensors within the urethra. Physicians typically adjust sling tension only using visual cues.

It is known to use a fluid pressure sensor located within the bladder to measure fluid pressure in the bladder. It is also known to insert a balloon between a sling and the urethra to measure compressive pressure upon the balloon by measuring the fluid pressure internal to the balloon.

### SUMMARY OF THE INVENTION

The present invention relates to sensing, measuring, and monitoring pressures in a tract or passage within a body (e.g., the urethra of a human female patient) such that diagnostic information is available to a physician or clinician performing a medical procedure (e.g., treatment of female urinary incontinence by use of a sling to elevate or support the bladder neck) on the body. The information could be acquired before, during, and/or following a surgical procedure.

A device according to the invention can be inserted into a urethra of a female patient for measuring pressure applied along a length of the urethra. A physician uses the device to obtain information about the pressure applied to a length of the urethra as a sling is manipulated and/or adjusted, and the device thus aids the physician in properly placing and situating the sling to best treat the urinary incontinence of the female patient.

In general, in one aspect, the invention involves a device for measuring pressure applied to a urethra. The device includes an elongate member that can be positioned within a lumen of a urethra. A plurality of pressure sensors are disposed on the elongate member. Each of the sensors is capable of outputting a signal that is representative of an amount of pressure applied to the urethra at the location of that sensor when the elongate member is positioned within the lumen of the urethra and pressure is applied to the urethra.

Embodiments of this aspect of the invention can include the following features. At least one of the signals output by the pressure sensors can be received by a display and subsequently displayed, thereby providing a representation of the amount of pressure represented by the signals. The representation of the amount of pressure may be in the form of a pressure distribution. The representation may, alternatively, be in the form of a two or three-dimensional display in which the pressure signals are measured radially around the circumference or axially along the length of a hemispherical sensor array. A sling disposed beneath the urethra can apply the pressure to the urethra. The plurality of pressure sensors can be electroactive pressure-sensitive film. The sensors can be a linear array or grid array. The center-to-center spacing between the sensors can be about 0.5 mm to about 5.0 mm. At least some of the sensors may be partially recessed into a surface of the elongate member. The pressure sensors can have a thickness of less than about 1.27 mm. The pressure sensors can include electrical leads that extend from the sensors and these leads can be partially located within the elongate member. Alternatively, a wireless transmitter can be used to transmit the signals from the pressure sensors. The pressure sensors can be capable of measuring pressure between about 0 psi and about 10 psi. The elongate member typically is sufficiently rigid to minimize deformation of the pressure sensors disposed on the elongate member when the elongate member is positioned within the lumen of the urethra.

In general, in another aspect, the invention relates to a method of determining pressure applied to a urethra by positioning an elongate member within a lumen of the urethra. The member includes sensors disposed thereon. When pressure is applied to the urethra at least some of the sensors output a signal representing the amount of applied pressure. A physician or other medical personnel can then perceive a representation of the applied pressure.

In general, in another aspect, the invention relates to a method of indicating pressure applied to a urethra. The method involves providing an elongate member with a plurality of pressure sensors disposed thereon. Signals output by the sensors are received when the member is positioned within the urethra and pressure is applied to the urethra, and a representation of the pressure is displayed.

This method can further include storing the signals output by the sensors in a computer system.

In general, in another aspect, the invention relates to a device for measuring pressure within a body of a patient. The device includes an elongate member that has a plurality of sensors disposed thereon. The device can be positioned within a body of a patient within a variety of locations, e.g., a bladder, a rectum, an esophagus, a vagina, a cervix, a tissue, a lumen, or a body cavity.

The foregoing and other objects, aspects, features, and advantages of the invention will become more apparent from the following description and from the claims.

### BRIEF DESCRIPTION OF THE DRAWINGS

In the drawings, like reference characters generally refer to corresponding parts throughout the different views. The drawings are not necessarily to scale, emphasis instead generally being placed on illustrating the principles and concepts of the invention.

FIG. 1 is an illustration of an embodiment of a urethral pressure sensor system of the present invention.

FIG. 2A is graphical illustration of a possible representation of pressure using a system of the invention such as the system of FIG. 1.

FIG. 2B is graphical illustration of a possible representation of pressure using a system of the invention such as the system of FIG. 1.

FIG. 2C is a graphical illustration of a possible representation of pressure using a system of the invention such as the system of FIG. 1.

FIG. 3A is an illustration of an embodiment of a sensor array according to the invention.

FIG. 3B is an illustration of an embodiment of a sensor array according to the invention.

FIG. 4 is an isometric view of an embodiment of an elongate member.

FIG. 5 is a perspective view of a pressure sensor array partially wrapped around an elongate member of the invention.

FIG. 6A is an exploded perspective view of an embodiment of a pressure sensor array coupled to an elongate member.

FIG. 6B is an assembly view of an embodiment of a pressure sensor array coupled to an elongate member.

FIG. 7 is a perspective view of a covering securing a pressure sensor array to an elongate member.

FIG. 8 is a partially broken-away view of an embodiment of a plurality of pressure sensors attached to an elongate member.

FIG. 9 is an illustration of an embodiment of a plurality of pressure sensors coupled to two elongate members that can move relative to each other.

FIG. 10A is a perspective view of a plurality of pressure sensors coupled to an embodiment of an elongate member.

FIG. 10B is a perspective view of a plurality of pressure sensors coupled to an embodiment of an elongate member.

FIG. 11A is an illustration of an embodiment of a sensor array and an elongate member.

FIG. 11B is an illustration of an embodiment of a sensor array coupled to an elongate member.

FIG. 12 is an illustration of some of the components of a wireless pressure sensor system.

FIG. 13A is an illustration of a sensor array coupled to an embodiment of an elongate member.

FIG. 13B is an illustration of a sensor array coupled to an embodiment of an elongate member.

FIG. 14A is a sagittal section of a female pelvis illustrating the location of a sling disposed beneath the urethra and a pressure sensing probe of the invention prior to insertion of the pressure sensing probe into the lumen of the urethra.

FIG. 14B is a sagittal section of a female pelvis illustrating the location of a sling disposed beneath the urethra and a pressure sensing probe of the invention following insertion of the pressure sensing probe into the lumen of the urethra.

### DESCRIPTION

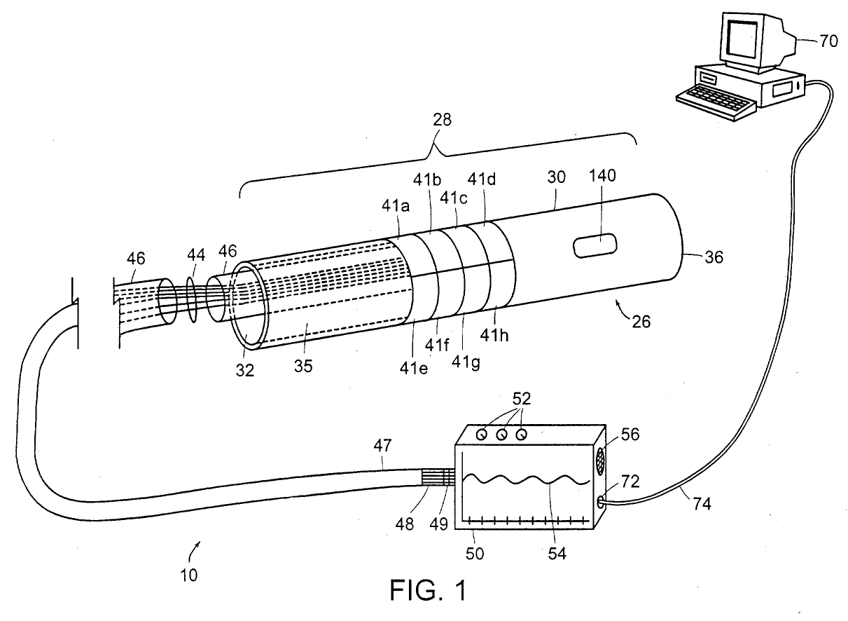

As shown in FIG. 1, one embodiment of a system 10 according to the invention includes a pressure-sensing probe 28 with eight pressure sensors 41a through 41h (generally 41) that are disposed on an elongate member 26 for measuring pressure applied to each of the pressure sensors 41a through 41h. In this embodiment, the visible surfaces of the pressure sensors 41a through 41h are located such that they are, generally, flush with the surface 30 of the elongate member 26. FIG. 1 also illustrates a series of wires 44 that are located within a jacket 46. The jacket 46 is located within a lumen 35 of the elongate member 26. The wires 44 are connected to the pressure sensors 41a through 41h. The wires 44 act as electrical leads to conduct electrical signals to (e.g., for providing electrical power to the sensors) and/or from (e.g., for conducting signals to a monitor or display) the pressure sensors 41a through 41h. The jacket 46 emerges from the elongate member 26 through an opening 32 in the elongate member 26. The jacket 46 and wires 44 could, alternatively, extend along the surface 30 of the elongate member 26. A proximal end 47 of the jacket 46 terminates with an electrical connector 48 which interfaces with a mating receptacle 49 located on a display 50. The display 50 displays a chart 54 that represents at least some of the data measured by the pressure sensors 41a through 41h, e.g., pressure at an individual sensor location, average pressure as measured by a user-defined subset of the pressure sensors 41a through 41h, or a distribution of pressure measurements as measured by the pressure sensors 41a through 41h.

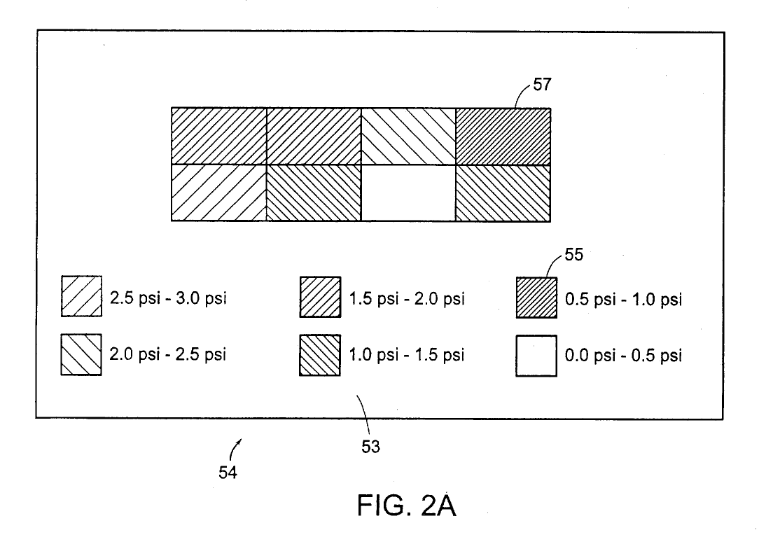

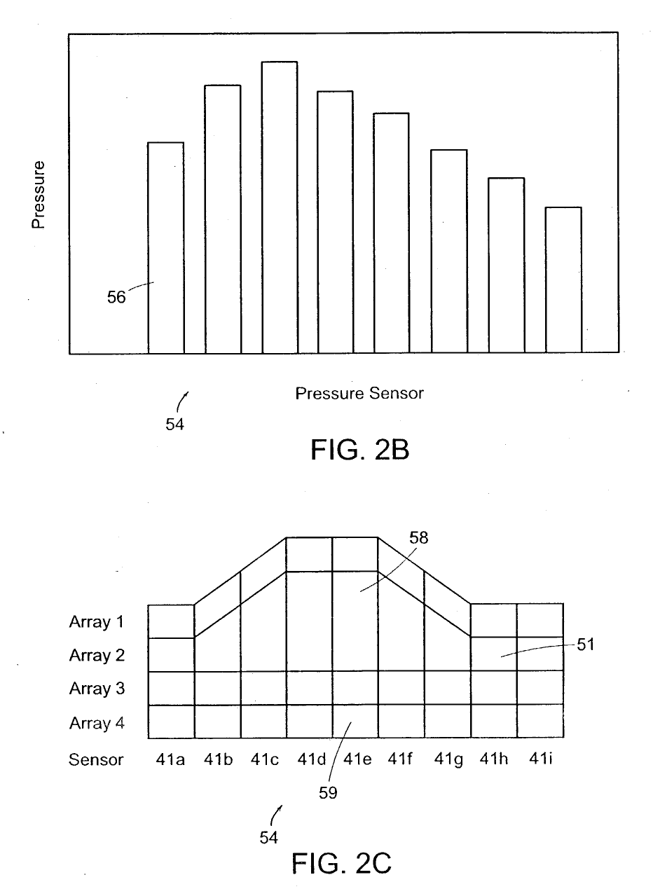

The chart 54 displayed by the display 50 can be configured, as illustrated in FIG. 2A, as a display of different pressure ranges 53. In this embodiment, a display element 57 is a graphical representation of the pressure measured by the pressure sensor 41d, of FIG. 1. The display element 57 provides a visual or graphical notification to an operator indicating, in this embodiment, that the value of the pressure measured by the pressure sensor 41d is between 0.5 psi and 1.0 psi, referring to a pressure range 55. The ranges 53 are illustrated as graphical patterns but could, alternatively, be represented by colors. In another embodiment, a chart 54 is a bar graph indicating relative levels of the pressure measured, e.g., a bar 56 corresponds to the pressure measured by the pressure sensor 41a, of FIG. 1.

In another embodiment, now referring to FIG. 2C, a chart 54 is a graphical representation of the pressure measured by each of nine pressure sensors (41a through 41i) for each of four arrays (Array 1 through Array 4). The size (e.g., area) of each display element 51 is proportional to the pressure measured by that respective pressure sensor. In this embodiment, the area defined by the rectangular region of a display element 58 is greater than the area defined by the rectangular region of a display element 59. As such, the pressure measured by the pressure sensor corresponding to the display element 58 is greater than the pressure measured by the pressure sensor corresponding to the display element 59.

Referring again to FIG. 1, several controllers 52 can control the output and operation of the display 50. Alternatively, a connection 72 located on the display 50 connects to a computer system 70 via a cable 74 and allows an operator to control the operation of the display 50. The electrical connector 48 could, alternatively, connect directly to the computer system 70. The computer system could then be used to display the data measured by the pressure sensors 41a through 41h. By way of example, the computer system 70 is capable of storing and recalling various data, and further manipulating (e.g., conducting a statistical analysis) the acquired data. This embodiment of the invention also comprises an audio speaker 56 capable of projecting an audio signal representative of some aspect of the pressure measured by the apparatus that can be perceived by the operator and used as diagnostic information in the medical procedure. In conjunction with (or instead of) listening to an aural indication of the pressure applied to the urethra, the operator can view a visual indication of the pressure applied to the urethra by using the display 50.

This embodiment of the invention also comprises a sensor 140 that is capable of measuring temperature of a portion of tissue in contact with the sensor 140, for example. One or more sensors could be attached to the elongate member 26 to allow a physician to monitor the temperature of a piece of tissue within a urethra during a cryotherapy medical procedure while the physician monitors the output of the plurality of pressure sensors 41a through 41h. This would enable the physician to monitor the temperature of the piece of tissue and vary the steps of the medical procedure to ensure the temperature remains below a predetermined threshold to ensure the safety of the patient.

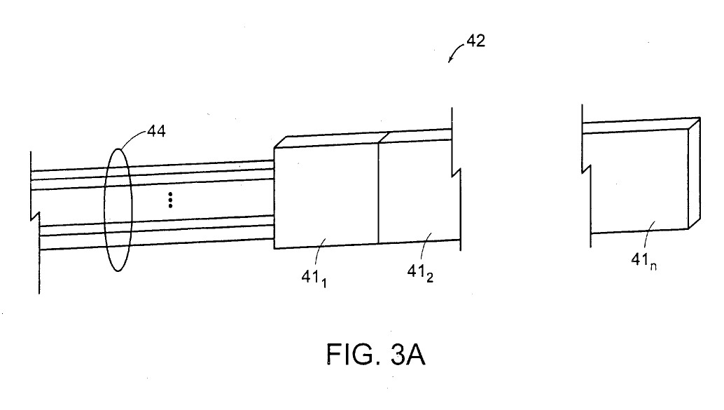

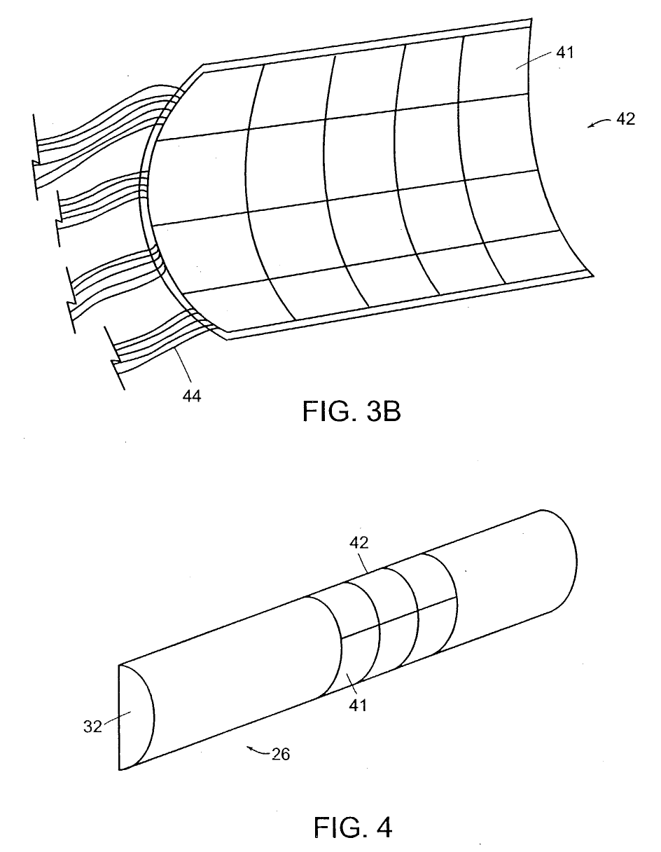

In an embodiment of the invention, as illustrated in FIG. 3A, a plurality of pressure sensors 41₁ through 41ₙ are mechanically coupled (e.g., with adhesive disposed between adjacent sensors) together in a sensor array 42 such that the pressure sensors 41₁ through 41ₙ can be handled as a generally unitary body and, as such, the sensor array 42 can be readily attached to a bar or rod in a single assembly step using, e.g., adhesive or glue. The sensor array 42 could, alternatively, be flat or curved to match the shape of a particular elongate member. By way of example, a flat sensor array might be coupled to a flat face of an elongate member. The choice of shape of the sensor array 42 as well as the choice of shape of the elongate member are, generally, dictated by the nature of the medical procedure an operator intends to perform (e.g., measuring the amount of pressure across a flat region of the body may require a flat sensor array coupled to a flat face of an elongate member). As illustrated in FIG. 3A, the wires 44 are in electrical communication with the pressure sensors 41₁ through 41ₙ such that each sensor has an individual set of electrical connections. In another embodiment of the invention, as illustrated in FIG. 3B, the pressure sensors 41 are configured as a grid in the sensor array 42.

By way of example, the sensor array 42 comprising the pressure sensors 41, referring now to FIG. 3B, might be manufactured using Xsensor® materials offered for sale by Sensor Products, Inc. with offices in East Hanover, N.J. Xsensor materials are electroactive pressure-sensitive films that behave similarly to an electrical capacitor. Xsensor materials generate an electrical signal in response to mechanical pressures or forces. The capacitive properties of the material change as a function of the pressure applied to the surface of the material. This change in capacitance is generally proportional to the applied change in pressure or force. The materials are generally capable of recording pressures between 0.1 pounds per square inch (psi) and 12,000 psi, however, typical pressures in an embodiment of the proposed invention used to measure intraurethral pressure are anticipated to be between about 0.0 psi and about 5.0 psi. The center-to-center spacing between sensors is typically between about 0.5 mm to about 5.0 mm. Further, the thickness of the sensor array 42 fabricated using Xsensor materials is typically about 0.48 mm, and generally less than about 1.27 mm.

In general, other types of sensors can be used to measure pressure in alternative embodiments of the invention. For example, resistive strain gages, piezoelectric (e.g., piezoresistive or piezocapacitive) materials (e.g., polymer or polycrystalline ceramic), or quartz pressure sensing devices can be used for each of the pressure sensors 41 of the sensor array 42. The alternative materials or devices could be bonded to an elongate member using an appropriate adhesive. Additionally, a combination of different sensors could be used in an alternative embodiment of the invention.

A plurality of pneumatic or hydraulic sensor elements could be used to fashion a sensor array that is disposed on the surface of or within an elongate member accompanied by, for example, wires used to communicate pressure signals to a display of the invention such as the display 50 of FIG. 1.

Optical fibers can also be used as sensors to measure pressure. The optical fibers could be bonded to the inside surface of an elongate member so that the fibers bend as a result of pressures being applied to the outside surface of the elongate member. A light signal passed through the fibers would change in response to the bending of the fiber. The change in the light signal would provide an operator with a measure of the change in pressure applied to, for example, a urethra as described herein.

The materials used to fabricate the pressure sensors 41 and sensor array 42 may be disposable or re-usable.

In another embodiment of one aspect of the invention, as illustrated in FIG. 4, the elongate member 26 comprises a hemispherical cross-section or other suitable geometric shape (e.g., ovate or rectangular) for measuring a 180 degree radial section of a location in a body.

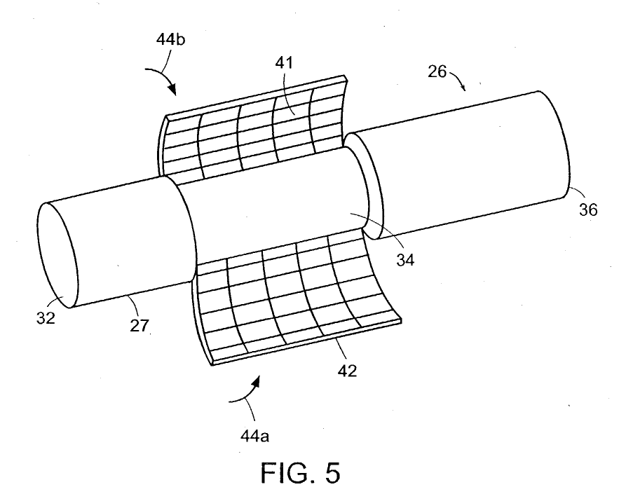

In an alternative embodiment of the invention, as illustrated in FIG. 5, a sensor array 42 is shown partially coupled to an elongate member 26. The sensor array 42 comprises a plurality of pressure sensors 41. The sensor array 42 is partially wrapped around a circumference of the elongate member 26 in a location 34 that has a smaller diameter than a section 27 of the elongate member 26. The sensor array 42 is flexible enough to wrap around the location 34, as illustrated by a set of arrows 44a and 44b, without breaking or tearing. The location 34 also serves as an alignment feature, allowing for the pressure sensors 41 to be, at least, partially recessed into a surface of the elongate member 26 during the manufacturing of the system. During fabrication, the sensor array 42 and, more specifically, each of the pressure sensors 41 are located at a pre-determined distance from an end 36 of the elongate member 26. The pre-determined distance may be chosen to ensure that the pressure sensors 41 are located a particular distance from an obstruction or wall that may abut the end 36 of the elongate member 26 when the sensor array 42 is being used to measure pressure.

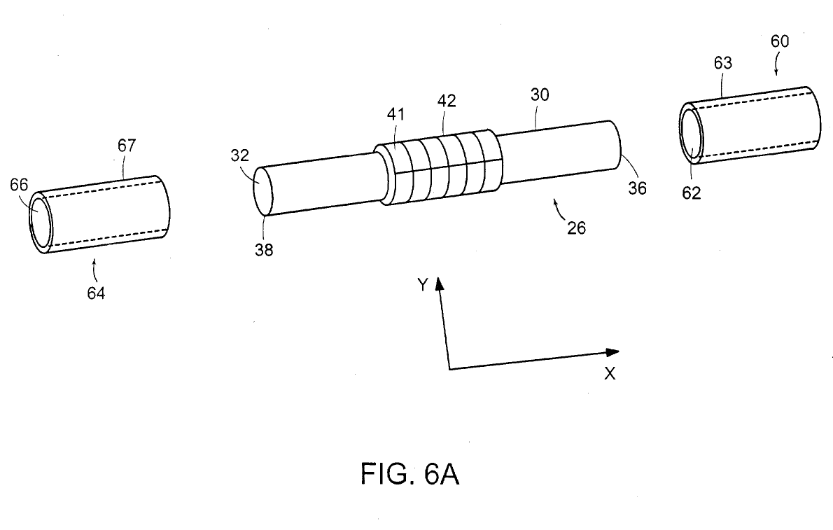

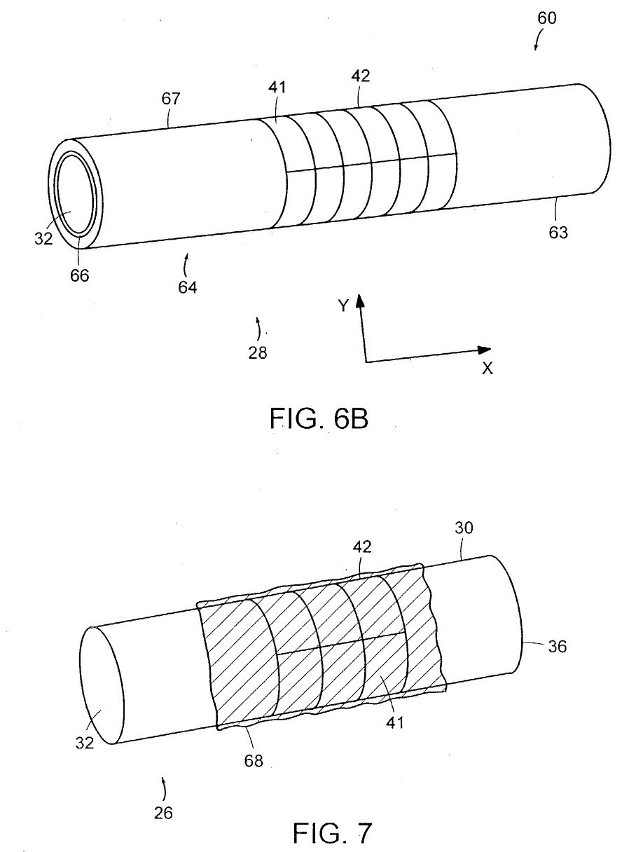

In another embodiment of the invention, as illustrated in FIG. 6A, a sensor array 42 is coupled to an elongate member 26. By way of example, the sensor array 42 might be coupled to the elongate member 26 using a structural epoxy disposed between the sensor array 42 and a surface 30 of the elongate member 26. The epoxy would, generally, function to limit linear motion of the sensor array 42 along the direction of the X-axis and rotational motion of the sensor array 42 around the X-axis.

FIG. 6A further illustrates two end pieces 60 and 64 that define two lumens 62 and 66, respectively. The end pieces 60 and 64 further comprise a set of outer surfaces 63 and 67, respectively. Referring now to FIG. 6B, the end pieces 60 and 64 are inserted over two ends 36 and 38, respectively, of the elongate member 26 in such a way as to ensure the outer surface of the sensor array 42 is roughly flush with the outer surfaces 63 and 67.

In an alternative embodiment, as illustrated in FIG. 7, a covering 68 (e.g., medically-approved polyurethane encapsulant, heat shrink tube, or biocompatible film) is placed over an elongate member 26 to fix the location of a sensor array 42 relative to the elongate member 26. The covering 68 applies a normal force (e.g., as the heat shrink tube shrinks) to a surface 30 of the elongate member 26 and a sensor array 42 to secure effectively the sensor array 42 in a predetermined location relative to an end 36 of the elongate member 26. Alternatively, adhesive disposed between the bottom of the sensor array 42 and the surface 30 could be used in conjunction with the covering 68 to secure the array 42 to the elongate member 26. The covering 68 could be a removable, disposable material that can be replaced between uses of a pressure-sensing probe according to the invention, such as the pressure-sensing probe 28 of FIG. 1.

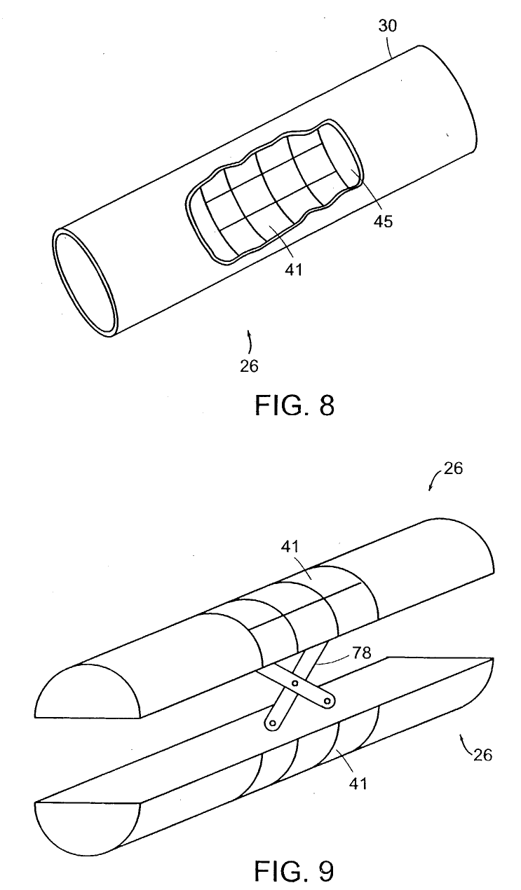

In an alternative embodiment, as illustrated in FIG. 8, a partially broken-away view of an elongate member 26 of the invention is provided in which a plurality of pressure sensors 41 are attached to an inner surface 45 of the elongate member 26. The pressure sensors 41 are sufficiently sensitive to pressures applied to an outer surface 30 of the elongate member 26 such that, e.g., the pressure measurements can be viewed by an operator using a display of the invention, such as the display 50 of FIG. 1.

In another embodiment, referring now to FIG. 9, two elongate members 26 are provided wherein each elongate member has a hemispherical cross-section. A plurality of pressure sensors 41 are coupled to the elongate members 26 for measuring pressure. The elongate members 26 are interconnected by a mechanism 78 that permits the elongate members 26 to translate or rotate relative to each other. This embodiment of a pressure-sensing probe system of the invention, such as the system 10 of FIG. 1, might be used by a physician to measure the pressure applied to the pressure sensors 41 by the inner walls of a vagina. This embodiment of the invention could be used in lieu of the physician inserting two of his or her fingers into the vagina of a female patient to determine the resistance to movement of the lateral walls of the vagina as the physician moves the two fingers apart from each other within the vagina. The resistance to movement may be, for example, indicative of a defect in the vaginal walls.

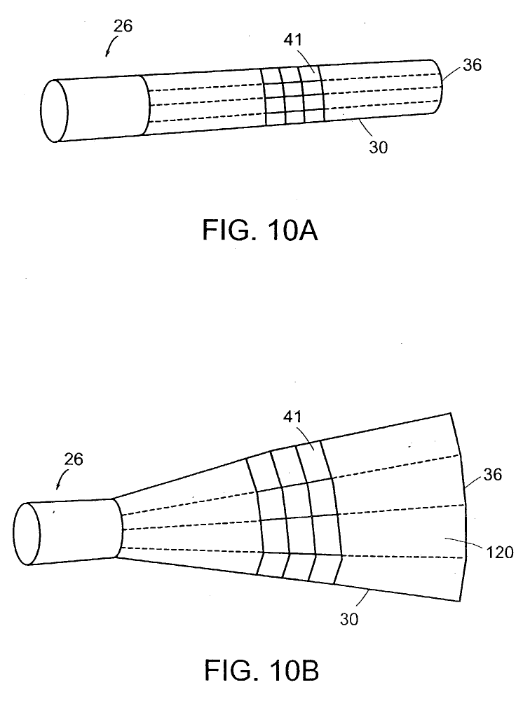

In another alternative embodiment, as illustrated in FIGS. 10A and 10B, a plurality of pressure sensors 41 are coupled to an elongate member 26. An end 36 of the elongate member 26 is initially compressed or retracted such that the outer diameter of the elongate member 26 is generally the same along the length of the elongate member 26. The end 36 then transforms into a flared or generally conical shape 120 such that an outer surface 30 of the elongate member 26 may conform to a flared or generally conical shaped lumen into which the elongate member 26 is inserted.

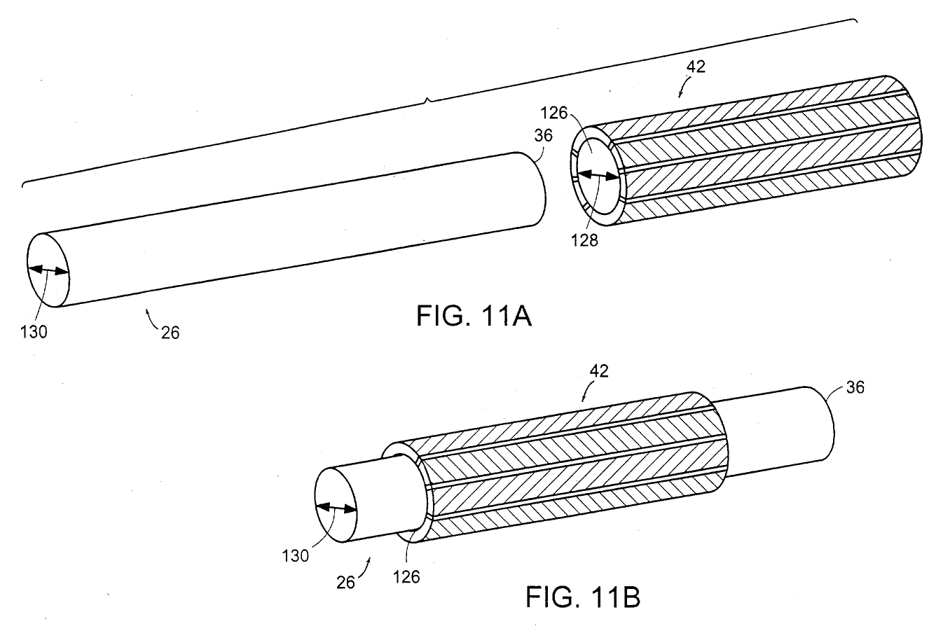

In an alternative embodiment, referring now to FIGS. 11A and 11B, an elongate member 26 with an outer diameter 130 is used to push apart or increase an inner diameter 128 of a cylindrical sensor array 42. In this embodiment, an end 36 of the elongate member 26 is inserted into a lumen 126 of the sensor array 42. The diameter 128 of the sensor array 42 increases to accommodate the larger outer diameter 130 of the elongate member 26 and to permit the elongate member 26 to pass through the lumen 126 of the sensor array 42. The sensor array 42 could, alternatively, be expanded in a radially outward direction by, for example, a cam driven mechanism. The cam driven mechanism can be inserted through the lumen 126 of the sensor array 42 and then subsequently triggered or actuated to increase the diameter 128 of the lumen 126 of the sensor array 42.

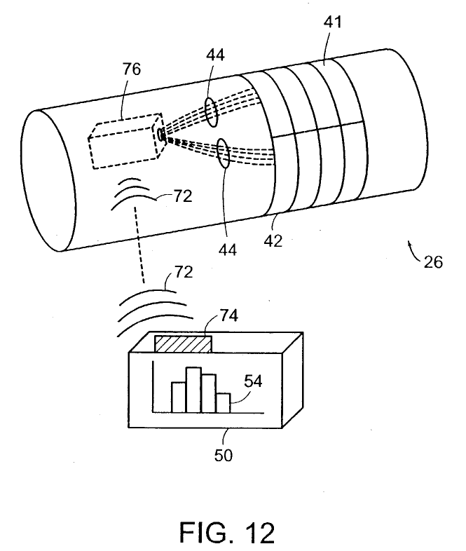

In another embodiment of the invention, as illustrated in FIG. 12, a wireless transmitter 76 is provided for transmitting at least one signal 72 to a receiver 74 located on a display 50. The display 50 is capable of displaying a chart 54 which is a representation of the pressure measurements provided by a plurality of pressure sensors 41 coupled to an elongate member 26. The pressure measurements are conducted by a set of wires 44 to the transmitter 76 and, subsequently, transmitted to the receiver 74. Additionally, the transmitter 76 could comprise a miniature, rechargeable battery for providing power to the transmitter 76 during use of, for example, a pressure sensing probe system of the invention, such as the system 10 of FIG. 1.

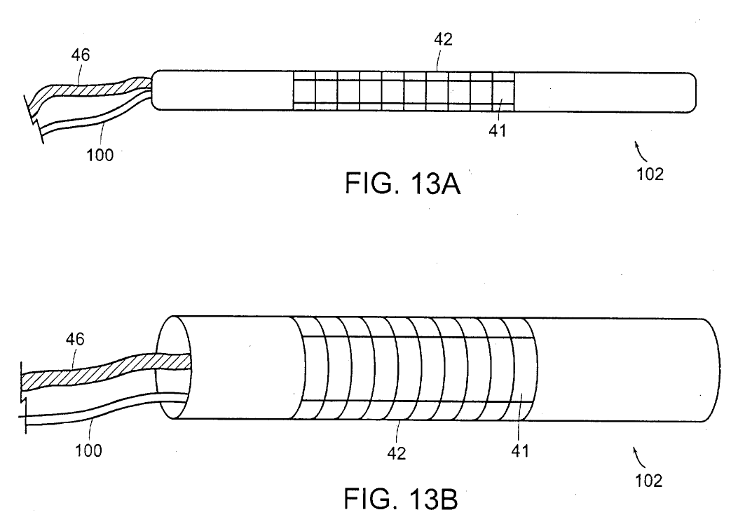

In another embodiment of an aspect of the invention, as illustrated in FIGS. 13A and 13B, a flexible elongate member 102 is capable of being inflated so that it assumes a rod shape. The elongate member 102 comprises a sensor array 42 and a jacket 46. Electrical leads, such as the wires 44 of FIG. 1, are located within the jacket 46 to provide an electrical connection between a plurality of pressure sensors 41 located on the sensor array 42 and, e.g., a display of the invention such as the display 50 of FIG. 1. This embodiment also comprises a tubular member that is connected to the flexible elongate member 102. The tubular member is capable of injecting or removing fluid (e.g., gas or liquid) from the flexible elongate member 102. In its non-inflated form, referring now to FIG. 13A, the flexible elongate member 102 can be passed through a lumen in a variety of common medical devices (e.g., a medical catheter) for delivery to a site internal to the body.

Once located in the body in close proximity to a desired location for monitoring pressure, fluid is injected into the flexible elongate member 102, referring now to FIG. 13B, via the tubular member thereby expanding the flexible elongate member 102. After fluid is injected into the flexible elongate member 102, the flexible elongate member 102 is sufficiently rigid and incompressible to ensure that the sensor array 42 and the pressure sensors 41 will not deform due to pressure applied to the sensor array 42 and the pressure sensors 41, e.g., by a urethra.

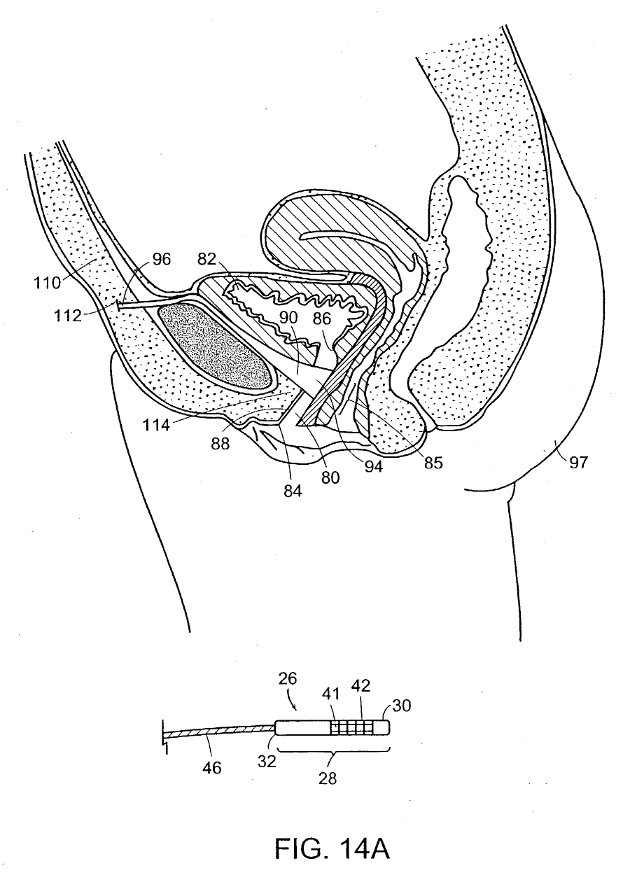

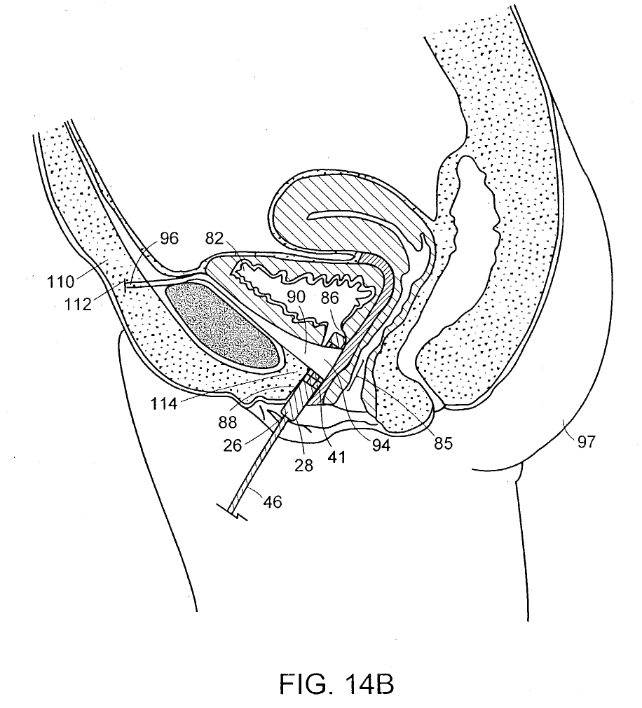

FIGS. 14A and 14B are sagittal sections of a female pelvis in a body 97. A bladder 82 located within the body 97 is connected to a urethra 80 that comprises an opening 84 in the body 97. A pressure-sensing probe 28 comprises a sensor array 42 attached to a surface 30 of an elongate member 26. A jacket 46 enters an opening 32 of the elongate member 26. Individual wires, such as the wires 44 of FIG. 1, are disposed within the jacket 46 and the wires are electrically connected to each pressure sensor 41 of the sensor array 42. FIGS. 14A and 14B further illustrate steps of an embodiment of the invention, in which the pressure-sensing probe 28 is guided into the opening 84 and co-axially along the urethra 80 such that the pressure-sensing probe 28 is, at least partially, disposed within the urethra 80. In this embodiment, the pressure-sensing probe 28 is used to measure pressure applied to the pressure sensors 41 that are disposed on the elongate member 26. The pressure-sensing probe 28 is of appropriate diameter and cross-sectional shape to ensure that at least a portion of a wall 88 of the urethra 80 at least partially contacts the outer surface of at least some of the pressure sensors 41. The pressure sensors 41 generate electrical signals representative of the pressure applied by the wall 88 on the pressure sensors 41 at the location of each individual sensor. The force applied by the wall 88 across the surface area of an individual pressure sensor 41 represents the pressure measured by that individual pressure sensor 41 (i.e., pressure=force/area). The generated electrical signals are also thus representative of the pressure being applied externally (by, for example, a sling 90) to the urethra.

Also illustrated in FIGS. 14A and 14B is the sling 90 that is used for treating urinary incontinence in female patients. The sling 90 is used to apply pressure to a mid-urethra region 85 of the urethra 80. By way of example, various urethral suspension procedures using a sling are described by Benderev et al. and shown in commonly-owned U.S. Pat. Nos. 5,860,425, and 6,077,216, which are hereby incorporated herein by reference. In one embodiment, the sling 90 is between about 2.0 cm and about 3.0 cm in width and is about 0.15 mm in thickness and manufactured from a woven mesh of polypropylene fiber. Alternatively, the sling 90 can be configured in different sizes and shapes and manufactured from other synthetic materials and/or biological tissues (e.g., cadaveric tissue).

In the disclosed embodiment a center portion 94 of the sling 90 is disposed below the mid-urethra region 85 of the urethra 80 with a sling end 96 extending upwards through muscle tissue (e.g., the endopelvic fascia and the rectus fascia) within the body 97 such that a sling end 96 is located at the location of an abdominal incision 112 in an abdomen 110 of the body 97. A matching sling end, not shown in this illustration, representing the opposite end of the sling 90 is located on the contra-lateral side of the body 97 and is located at the location of an abdominal incision in the abdomen 110 on the contra-lateral side of the body 97. The sling end 96 and its matching opposite end can be manipulated or positioned so that the center portion 94 of the sling 90 applies pressure to the urethra 80 or raises the urethra 80 within the body 97. The sling end 96 and its matching opposite end are positioned to increase or decrease pressure to the urethra 80 as determined, generally, by a physician. The physician can determine whether to increase or decrease the pressure applied by the sling 90 to the urethra 80 by observing (e.g., audibly and/or visually) the output of pressure-sensing probe 28 using a display of the invention, such as the display 50 of the system of FIG. 1.

The pressure applied by the sling 90 to the mid-urethra region 85 of the urethra 80 could be specified by a doctor to result in the mid-urethra region 85 to be repositioned to minimize involuntary or undesirable urine expulsion from the bladder 82 through the urethra 80 and out of the urethral opening 84. Subsequent to repositioning the mid-urethra region 85, the physician would close the incisions 112 in the body 97. Muscle tissue (e.g., endopelvic fascia and rectus fascia) in contact with the sling 90 secure and maintain the position of the sling 90 in the body 97, thereby maintaining the pressure applied by the sling 90 on the mid-urethra region 85 of the urethra 90. The pressure applied by the sling 90 on the mid-urethra region 85 minimizes the likelihood of undesirable expulsion of urine from the bladder. In an alternative embodiment, (e.g., in a bladderneck procedure) the physician could attach the sling end 96 to the superior end of the pubic bone using a bone anchor or otherwise suitably secure the location of the sling end 96 and the tension of the sling 90, thereby minimizing the likelihood of undesirable expulsion of urine from the bladder.

A bulking agent can be used to treat intrinsic sphincter deficiency in a female urethra. By way of example, the bulking agent might be a natural material (e.g., a collagen material) or a synthetic material (e.g., pyrolitic carbon coated beads contained in a water based carrier gel sold under the product name Durasphere® by Carbon Medical Technologies, Inc. with offices in St. Paul, Minn.). The bulking agent is injected into sphincter tissue within the urethra to expand or bulk up the tissue, thereby decreasing the size of the lumen defined by the sphincter tissue. The bulking agent is typically injected until the tissue walls apply pressure to each other thereby achieving a fluid seal to prevent unwanted expulsion of urine. During the procedure, the physician must determine how much or how little the tissue of the sphincter should be bulked up so that appropriate sphincter closing pressure is achieved. Too much closing pressure results in a situation where the patient may find it difficult to urinate because her muscles are not strong enough to counter the closing pressure. Alternatively, if too little closing pressure is developed urine may still leak from the patient. Further, the bulking agent used for bulking is typically injected into the sphincter tissue in several locations around the circumference of the sphincter and along the length of the sphincter thereby affecting the closing pressure both radially around a circumference of the sphincter and axially along the length of the sphincter.

This procedure is typically performed by a physician based upon visual cues (e.g., sphincter tissue contracting) and experience. A pressure-sensing probe system of the invention, such as the system 10 of FIG. 1, would be capable of providing a physician with pressure measurements to gauge the closing pressure. The system 10 would provide a physician with pressure measurements radially around the circumference of the inside lumen of the sphincter as well as longitudinally along the length of the sphincter. The system 10 would be capable of generating a plurality of pressure measurements, and as such, the system 10 would be capable of providing the physician with the ability to assess the closing pressure of the sphincter at various locations within the sphincter.

Alternative embodiments of the invention are contemplated for measuring pressure applied, e.g., by a body organ or cavity to the sensors disposed on the elongate member. Various aspects of the invention would be modified (e.g., diameter of the cross-section of the elongate member, center-to-center spacing between each of the sensors, or the shape of the elongate member) in accordance with the requirements associated with measuring pressure at a particular location in the body.

Alternative embodiments of the invention are contemplated for measuring pressure within the urinary bladder (i.e., within the detruser muscle) during neurostimulation for the treatment of urge incontinence, in the cervix during dilation measurement and assessment, and in a passage between the bladder and the ureter during measurement and assessment of urinary reflux. Further, pressure measurements can be acquired at the vesicular junction of the urethra for occlusion force assessment, within the urethra to assess urethral stricture that may be caused by inflammation or scar tissue resulting from urethral surgery, and within the esophageal sphincter during treatment of gastroesophageal reflux disease.

Variations, modifications, and other implementations of what is described herein will occur to those of ordinary skill without departing from the spirit and the scope of the invention. Accordingly, the invention is not to be defined only by the preceding illustrative description.

What is claimed is:

1. A device for measuring pressure applied to a urethra, comprising:
an elongate member positionable within a lumen of the urethra; and
a plurality of pressure sensors disposed on the elongate member, each of the plurality of pressure sensors capable of outputting a signal representative of an amount of pressure applied to the urethra at the location of that sensor when the elongate member is positioned within the lumen of the urethra and pressure is applied to the urethra.

2. The device of claim 1 further comprising a display for receiving at least one of the signals and displaying a representation of the amount of pressure represented by the at least one signal.

3. The device of claim 2 wherein the representation displayed by the display is a pressure distribution.

4. The device of claim 1 wherein a sling disposed beneath the urethra applies pressure to the urethra.

5. The device of claim 1 wherein the plurality of pressure sensors comprises an electroactive pressure-sensitive film disposed around the elongate member.

6. The device of claim 1 wherein the plurality of pressure sensors comprises a linear array of the pressure sensors.

7. The device of claim 6 wherein the center-to-center spacing between each of the sensors in the linear array is between about 0.5 mm and about 5.0 mm.

8. The device of claim 1 wherein the plurality of pressure sensors comprises a grid array of the pressure sensors.

9. The device of claim 8 wherein the center-to-center spacing between each of the sensors in the grid array is between about 0.5 mm and about 5.0 mm.

10. The device of claim 8 wherein the grid array is disposed at least partially around a circumference of the elongate member.

11. The device of claim 1 wherein at least some of the plurality of pressure sensors are partially recessed into a surface of the elongate member.

12. The device of claim 1 wherein each of the plurality of pressure sensors has a thickness of less than about 1.27 mm.

13. The device of claim 1 wherein each of the plurality of sensors includes at least one electrical lead extending from the sensor.

14. The device of claim 13 wherein the electrical leads are located at least partially within the elongate member.

15. The device of claim 1 wherein each of the plurality of pressure sensors is capable of measuring pressure between about 0 psi and 5 psi.

16. The device of claim 1 wherein the elongate member is sufficiently rigid to minimize deformation of the plurality of pressure sensors disposed on the elongate member when the elongate member is positioned within the lumen of the urethra and pressure is applied to the urethra.

17. The device of claim 1 further comprising a wireless transmitter coupled to at least one of the plurality of pressure sensors for transmitting the signal output by that sensor when the elongate member is positioned within the lumen of the urethra and pressure is applied to the urethra.

18. A method of determining pressure applied to a urethra, comprising:
providing an elongate member including a plurality of pressure sensors disposed on the elongate member, each of the plurality of pressure sensors capable of outputting a signal representative of an amount of pressure applied to that sensor;
positioning the elongate member, with the sensors disposed thereon, within a lumen of the urethra;
applying pressure to the urethra such that each of at least some of the plurality of pressure sensors outputs the signal representative of the amount of pressure applied to that sensor; and
perceiving a representation of the amount of pressure represented by the at least some of the signals to determine pressure applied to the urethra.

19. The method of claim 18 wherein the step of applying pressure comprises applying pressure to the urethra by manipulating a sling disposed beneath the urethra.

20. The method of claim 18 wherein the step of perceiving comprises viewing the representation which comprises a visual indication of the amount of pressure.

21. The method of claim 18 wherein the step of perceiving comprises listening to the representation which comprises an aural indication of the amount of pressure.

22. A method of indicating pressure applied to a urethra, comprising:
providing an elongate member including a plurality of pressure sensors disposed on the elongate member, each of the plurality of pressure sensors capable of outputting a signal representative of an amount of pressure applied to that sensor;
receiving the signals from at least some of the sensors when the elongate member is positioned within a lumen of the urethra and pressure is applied to the urethra; and
displaying a representation of the amounts of pressure applied to the at least some of the sensors.

23. The method of claim 22 further comprising:
providing a computer system capable of storing the signals output by the at least some of the sensors; and
storing the signals output by the at least some of the sensors with the computer system.

24. A device for measuring pressure within a body of a patient, comprising:
an elongate member positionable in the body; and
a plurality of pressure sensors disposed on the elongate member, each of the plurality of pressure sensors capable of outputting a signal representative of an amount of pressure applied to the sensor when the elongate member is positioned within the body;

25. The device of claim 24 wherein the elongate member is positionable within a bladder of the body.

26. The device of claim 24 wherein the elongate member is positionable within a rectum of the body.

27. The device of claim 24 wherein the elongate member is positionable within an esophagus of the body.

28. The device of claim 24 wherein the elongate member is positionable within a vagina of the body.

29. The device of claim 24 wherein the elongate member is positionable within a cervix of the body.

30. The device of claim 24 wherein the elongate member is positionable within a tissue in the body.

31. The device of claim 24 wherein the elongate member is positionable within a body cavity.

32. The device of claim 24 wherein the elongate member is positionable within a lumen of the body.
